(19) United States
(12) Patent Application Publication
(10) Pub. No.: US 2026/0074039 A1
(43) Pub. Date: Mar. 12, 2026

(54) Method of Data Analysis for Long-Term Blood Glucose Concentration Trend

(71) Applicant: National Taiwan University, Taipei (TW)

(72) Inventors: Chi-Kuang Sun, Taipei (TW); Xu-hao Ye, Taipei (TW); Tzung-Dau Wang, Taipei (TW)

(21) Appl. No.: 19/389,368

(22) Filed: Nov. 14, 2025

Related U.S. Application Data
(63) Continuation-in-part of application No. 18/113,692, filed on Feb. 24, 2023.

(30) Foreign Application Priority Data
Jan. 18, 2023 (TW) 112102256

(51) Int. Cl. G16H 10/60; G16H 40/00

(52) U.S. Cl. CPC G16H 10/60; G16H 40/00

(57) ABSTRACT

A method of data analysis is provided. The method is used for finding a long-term trend of blood glucose concentration. The method builds a model for estimating long-term glycemic variability and long-term blood glucose trajectory. Based on single-erythrocyte-level glycated hemoglobin distribution, the glycemic variability is analyzed. A first analysis method is to give a number. The number shows the level of the historical glycemic variabilities. A second analysis method is to restore the blood glucose trajectory over the past 20 weeks. Based on the single-erythrocyte-level glycated hemoglobin distribution, the present invention easily assesses blood-glucose-related clinical information for about 150 days. Hence, an important complement is obtained for diabetes-related or glucose-monitoring-related clinical applications.

### TECHNICAL FIELD OF THE INVENTION

The present invention relates to the analysis of blood glucose concentration and, more particularly, to a method and system for analyzing glycemic variability and reconstructing historical blood glucose trajectories based on single-erythrocyte-level glycated hemoglobin distributions.

### DESCRIPTION OF THE RELATED ARTS

All existing glycated-hemoglobin-based glucose-estimation methods cannot provide any information regarding blood glucose fluctuations although can assess ˜3-month average blood glucose level. Evolving evidence indicates that variations in blood glucose levels are associated with diabetes-related complications independent of glycated hemoglobin fraction.

Devices such as continuous blood glucose monitoring allow people with diabetes to determine their blood glucose levels on a continuous basis and estimate blood glucose fluctuations. However, the sensors must be changed about every 2 weeks, and the cost related could limit their widespread use.

All these methods could not provide long-term blood-glucose-related information instantly. Hence, the prior arts do not fulfill all users' requests on actual use.

### SUMMARY OF THE INVENTION

The present invention provides a method and system to overcome the limitations of the prior art. Embodiments of the invention analyze a subject's historical glycemic status by processing a distribution of single-cell glycated hemoglobin measurements.

In one aspect, an embodiment of the invention provides a method for reconstructing a subject's historical glucose concentration trajectory, G(τ), from single-cell glycated hemoglobin (HbA1c) measurements. The method comprises: (a) obtaining a plurality of single-red-blood-cell (RBC) HbA1c fractions, {Aᵢ}, from a total of N RBCs sampled from a subject; (b) generating a first RBC HbA1c distribution, PDF_{HBA1c}(A), from said plurality of {Aᵢ}, represented as a histogram with a selected bin width ΔA; (c) defining a whole-blood RBC age probability density function, PDF_{Age}(t); (d) defining an RBC glycation model, A(t), that provides a deterministic mapping between the historical glucose concentration trajectory, G(τ), and a corresponding HbA1c fraction for an RBC of age t; (e) determining a time resolution ΔT corresponding to the bin width ΔA; (f) generating a reference RBC HbA1c distribution establishing an initial candidate glucose trajectory, G(τ), solving the glycation model A(t) based on said candidate G(τ), and computationally transforming said PDF_{Age}(t) into the reference distribution using the mapping from A(t); (g) iteratively modifying the candidate G(τ) by calculating a deviation metric between the first and reference distributions, adjusting G(τ) to create a new candidate G(τ), and repeating until the deviation metric is below a threshold; and (h) outputting the final candidate G(τ) as the reconstructed trajectory.

In another aspect, an embodiment of the invention provides a method for assessing a subject's glycemic variability (GV). The method comprises: (a) obtaining a plurality of single-RBC HbA1c fractions, {Aᵢ}; (b) generating a first RBC HbA1c distribution, PDF_{HBA1c}(A); (c) defining a whole-blood RBC age probability density function, PDF_{Age}(t); (d) defining an RBC glycation model, A(t); (e) determining an estimated average glucose (eAG) level for the subject; (f) generating a second RBC HbA1c distribution by establishing a stable glucose trajectory (wherein G(τ) is held constant at said eAG), solving the glycation model A(t), and computationally transforming said PDF_{Age}(t) into said second distribution; (g) calculating a deviation metric representing an absolute deviation between said first and second distributions; and (h) outputting said deviation metric as an indicator of the subject's glycemic variability.

In another aspect, an embodiment of the invention provides a system for analyzing a subject's historical glycemic status. The system comprises: (a) a data processing terminal comprising one or more processors; and (b) a non-transitory machine-readable medium storing instructions that, when executed, cause said processors to: (i) receive a first RBC HbA1c distribution, PDF_{HBA1c}(A), generated from a plurality of HbA1c fractions, {Aᵢ}; (ii) define a whole-blood RBC age probability density function, PDF_{Age}(t); (iii) define an RBC glycation model, A(t); and (iv) generate a subsequent RBC HbA1c distribution (such as a reference distribution or a stable-glucose distribution) based on said PDF_{Age}(t) and said A(t). The system may be further configured to perform the full methods of trajectory reconstruction or GV assessment.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according the present invention;

FIG. 2A˜FIG. 2O are the views showing the blood glucose trajectories corresponding to the glycated hemoglobin distributions, separately;

FIG. 3A˜FIG. 3F are the estimation views showing the long-term glycemic variabilities; and

FIG. 3G˜FIG. 3H are the estimation view showing the long-term glycemic variability and the long-term reconstructed blood glucose trajectory of the state-of-use.

### DESCRIPTION OF THE PREFERRED EMBODIMENT

The following description of the preferred embodiment is provided to understand the features and the structures of the present invention.

Embodiments of the invention provide a method and system for analyzing historical glycemic status. The system may be embodied as a data processing terminal, such as a general-purpose computer, a specialized medical device, or a cloud-based server, comprising one or more processors and a non-transitory machine-readable medium (e.g., memory, hard drive). This medium stores instructions that, when executed by the processors, perform the methods described herein. The system may further include a data acquisition interface configured to receive data from an external assay or instrument.

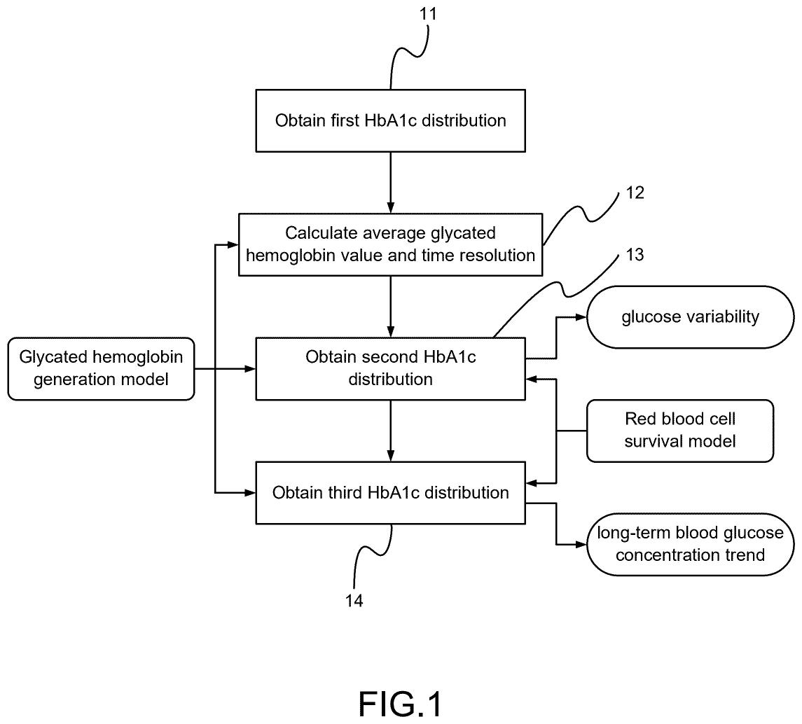

FIG. 1 shows a flowchart of the method. The process begins at step 11, “Obtain first HbA1c distribution”. This distribution, PDF_{HBA1c}(A), is the primary input data, generated from a plurality of single-RBC HbA1c measurements from an examinee.

From this, two main analyses can be performed, both of which require two core biophysical models: a “Glycated hemoglobin generation model” (i.e., A(t)) and a “Red blood cell survival model” (i.e., PDF_{Age}(t)).

In a first analysis path, the method proceeds to step 12, “Calculate average glycated hemoglobin value and time resolution”. Then, in step 13, “Obtain second HbA1c distribution”, this average value is used to generate a stable, theoretical distribution. This second distribution is compared to the first distribution (from step 11) to compute a “glucose variability” (GV) metric.

In a second analysis path, the method proceeds from step 12 to step 14, “Obtain third HbA1c distribution”. Here, a candidate historical trajectory is used to generate a reference (third) distribution. This third distribution is compared to the first distribution (from step 11). The candidate trajectory is iteratively modified to minimize the fitting error, ultimately yielding a “long-term blood glucose concentration trend”.

Each component of this process will now be described in detail.

### 1. DATA ACQUISITION AND FIRST DISTRIBUTION

The method begins by obtaining a plurality of single-red-blood-cell (RBC) HbA1c fractions, {Aᵢ}, from a total of N RBCs sampled from a subject (Claims 1(a), 2(a)). Each fraction Aᵢ represents the ratio of the glycated hemoglobin amount to the total hemoglobin amount within that single RBC. This data may be received by the system's data acquisition interface (Claim 17).

This step (a) can be performed by any assay or technique capable of determining the HbA1c fraction within a single RBC (Claim 12). Non-limiting examples of such techniques include High-Performance Liquid Chromatography (HPLC), capillary electrophoresis, immunoassay, mass spectrometry, Raman-based methods, transient absorption microscopy, absorption spectroscopy, or other optical microscopy techniques.

In a preferred embodiment (Claim 13), the plurality of {Aᵢ} is obtained using Resonant-Enhanced Color-Resolved Third-Harmonic-Generation (RE-cTHGM) microscopy. This non-invasive optical technique is based on the discovery that hemoglobin (Hb) and glycated hemoglobin (HbA1c) have distinct absorption spectra in the Soret band (approx. 415 nm), with HbA1c's absorption peak being slightly red-shifted (˜2 nm) compared to Hb.

The RE-cTHGM method comprises:
(a) Exciting a single RBC with a single broadband laser beam (e.g., from a Cr:forsterite laser centered near 1260 or a nm) wavelength-tunable laser beam.
(b) Generating a color-resolved third-harmonic-generation (THG) signal from the RBC. The THG signal is resonantly enhanced by the three-photon resonance with the Soret band absorption, meaning the subtle 2-nm spectral shift between Hb and HbA1c is amplified into a more pronounced, measurable shift in the generated THG spectrum (which appears at ⅓ the excitation wavelength, e.g., ˜410-420 nm).
(c) Partitioning this resonantly enhanced THG signal into at least two distinct spectral channels using dichroic mirrors and band-pass filters.
(d) Computing the HbA1c fraction Aᵢ for that single RBC from a relative signal intensity between the at least two spectral channels. For example, a first channel (e.g., “Channel A,” ˜410-415 nm) is selected to be more sensitive to non-glycated hemoglobin (Hb), and a second channel (e.g., “Channel B,” ˜415-420 nm) is selected to be more sensitive to glycated hemoglobin (HbA1c). The HbA1c fraction Aᵢ is then computed from the ratio of these channels (e.g., a “B/A ratio”).

This RE-cTHGM instrument may be operatively coupled to the system's data acquisition interface to provide the {Aᵢ} data in real-time (Claim 18).

Once the plurality of {Aᵢ} is obtained, the method generates a first RBC HbA1c distribution, PDF_{HBA1c}(A) (Claims 1(b), 2(b); FIG. 1, step 11). This is typically represented as a histogram, grouping the N measurements into bins of a selected bin width ΔA.

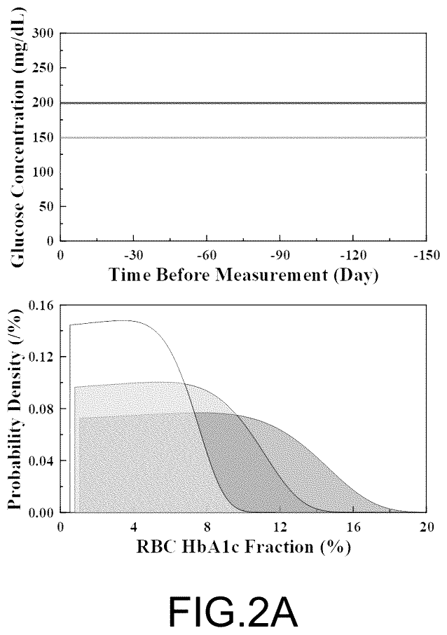

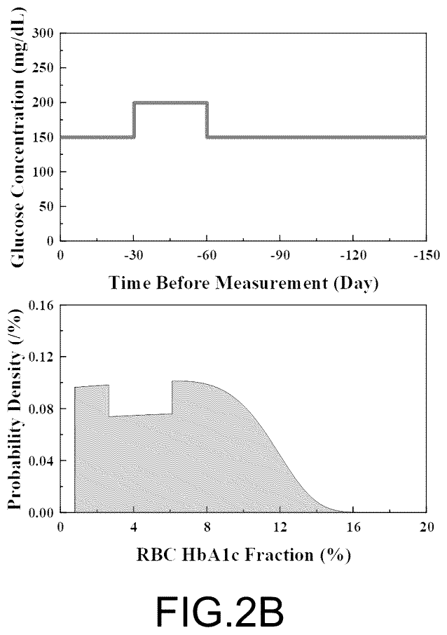

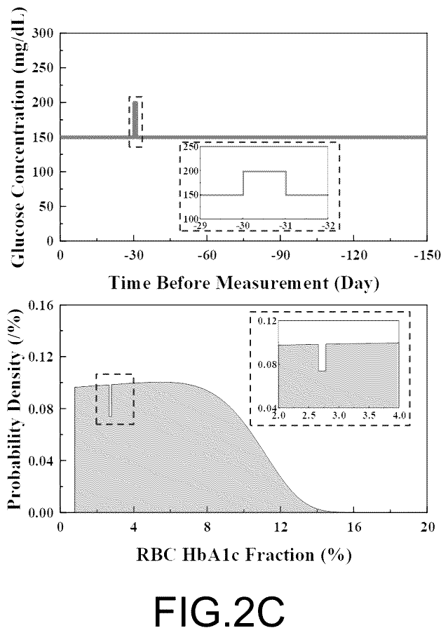

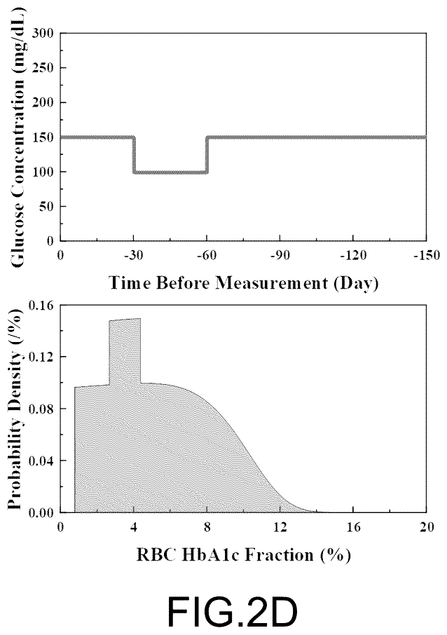

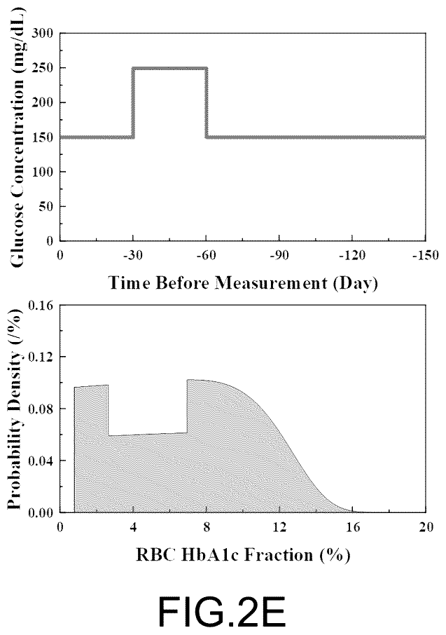

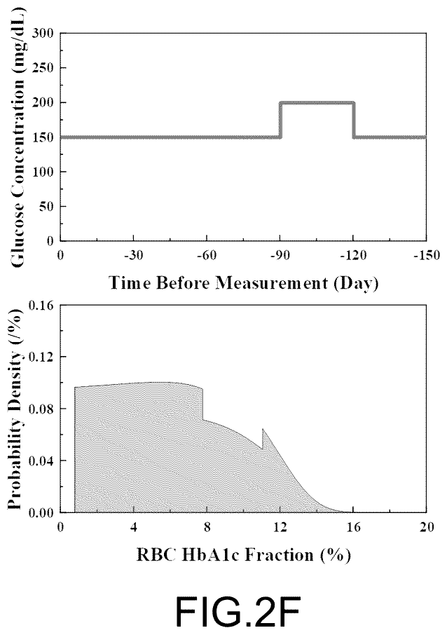

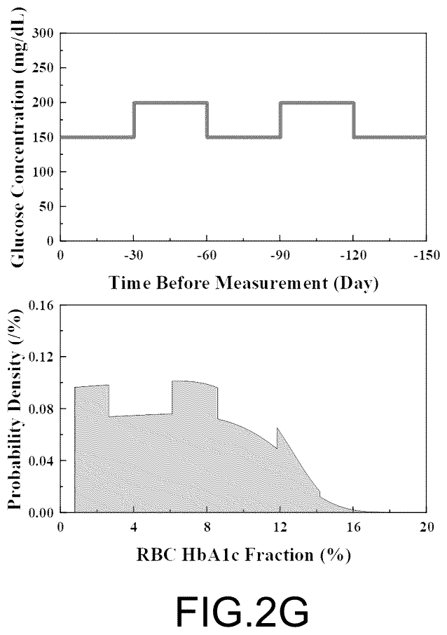

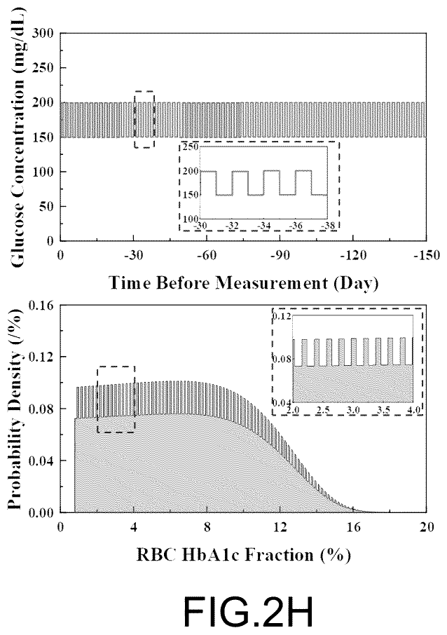

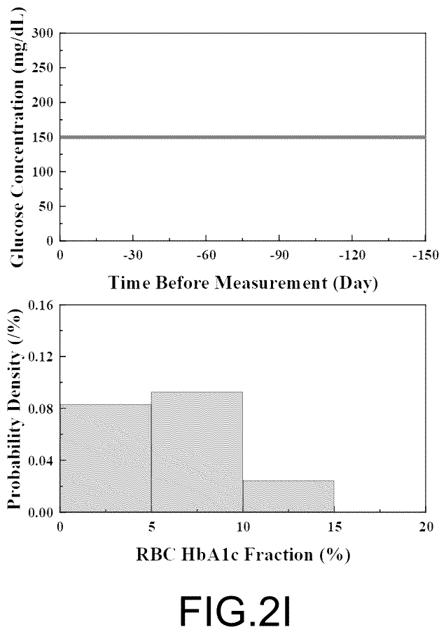

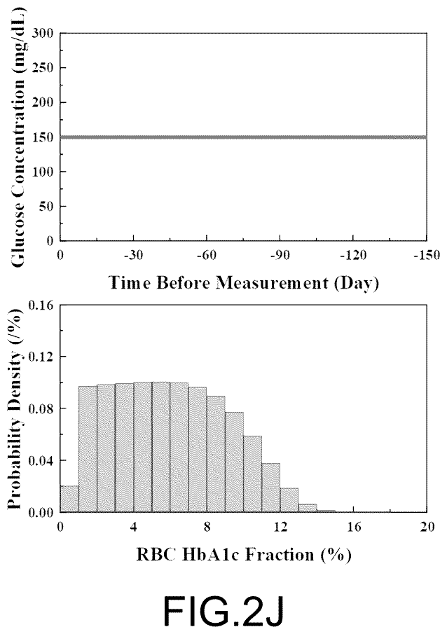

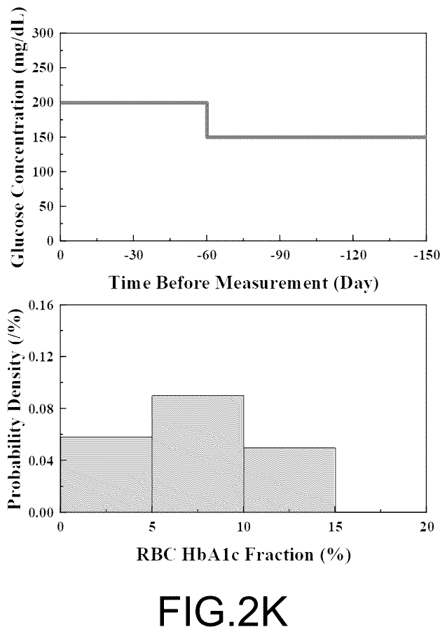

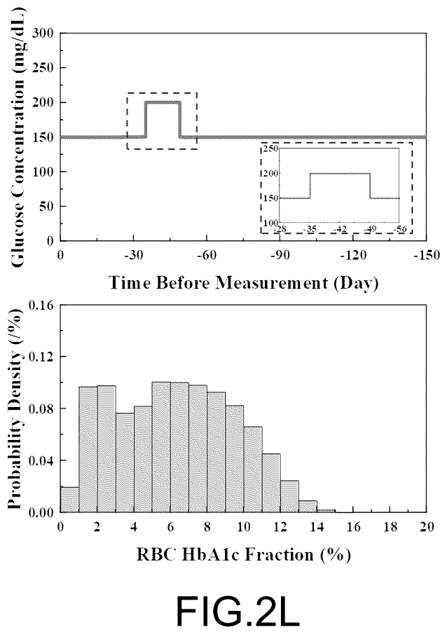

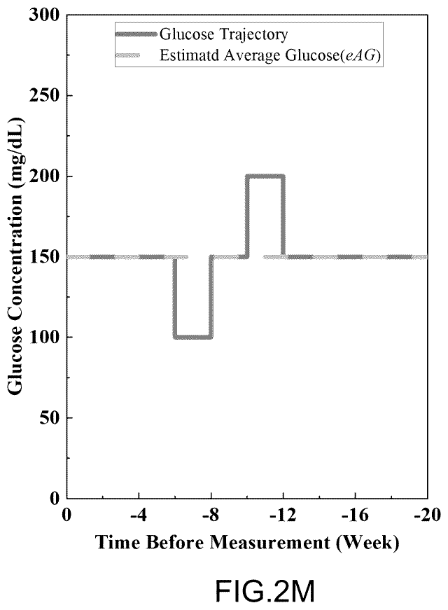

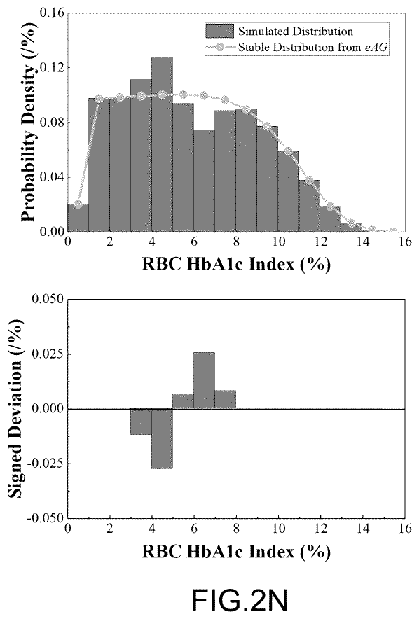

The choice of ΔA is dependent on the total number of cells N (Claim 8). A smaller N requires a larger ΔA to ensure statistical significance in each bin, while a larger N permits a smaller ΔA. The bin width ΔA may be determined by applying a statistical histogram binning rule, such as, but not limited to, the Freedman-Diaconis rule or Scott's rule. This is illustrated in FIG. 2I (a coarse histogram with wide ΔA from a small N) and FIG. 2J (a finer histogram with narrow ΔA from a larger N).

This bin width ΔA directly corresponds to the achievable time resolution ΔT of the reconstructed trajectory (Claims 1(e), 2(h), Claim 9). A smaller ΔA (higher HbA1c resolution) allows for a smaller ΔT (higher temporal resolution). The time resolution ΔT is determined as a function of ΔA and the estimated glycation rate, dA/dt, which is derived from the RBC glycation model A(t) and the glucose trajectory G(τ).

### 2. CORE BIOPHYSICAL MODELS

The method relies on two core biophysical models defined by the system.

A. RBC Age Distribution, PDF_{Age}(t) (Claims 1(c), 2(c), 6)

The method defines a whole-blood RBC age probability density function, PDF_{Age}(t), which represents the statistical distribution of RBCs having a circulation age t (where t=0 is the time of release from bone marrow) (Claims 1(c), 2(c)). This function is also referred to as the “Red blood cell survival model” in FIG. 1.

This function may be defined by various methods (Claim 6). In one embodiment, it is determined by direct measurement of RBC ages from the subject. In a preferred embodiment, PDF_{Age}(t) is reconstructed by applying a mathematical survival function model to a measured survival rate. This survival rate is obtained by: (i) administering a tagged cohort of RBCs (e.g., tagged with biotin or a radioactive element such as ⁵¹Cr) to the subject, (ii) sampling blood at multiple different time points, and (iii) measuring the change in the tagged RBC population over time to determine the survival curve. This method is effective because no new, unmarked RBCs are recruited into the tagged cohort after re-infusion. Therefore, after a time interval T, the age distribution of the tagged cohort, which began as a whole-blood age distribution, is now missing the 0-T age group, allowing for the reconstruction of the age distribution.

In a specific, non-limiting example, this survival function is modeled as a Weibull survival distribution,

$$W ( t ) = {e}^{- {( \frac{t}{b} )}^{a}} .$$

The corresponding PDF_{Age}(t) is then derived from this survival function. Based on published literature, exemplary parameters for a healthy human subject are a=5.58 and b=125.63 days.
B. RBC Glycation Model, A(t) (Claims 1(d), 2(d), 3, 4, 5)

The method also defines an RBC glycation model, A(t), which provides a deterministic mapping between a given historical glucose concentration trajectory, G(τ), and the corresponding HbA1c fraction A for an RBC of a specific age t (Claims 1(d), 2(d)). This function, A(t), is referred to as the “Glycated hemoglobin generation model” in FIG. 1 and is the critical link used to computationally transform the known age distribution, PDF_{Age}(t), into a theoretical HbA1c distribution, PDF_{HbA1c}(A).

In a preferred embodiment (Claim 3), this glycation model A(t) is defined by the integral equation:

$$A ( t ) = {\int }_{- {T}_{0}}^{t} {k}_{g} * G ( τ ) * ( 1 - A ( τ ) ) d τ .$$

Here, k_{g} is a hemoglobin glycation reaction constant, G(τ) is the glucose concentration at time τ, and T₀ is a pre-circulation residence time in the bone marrow. The model assumes an initial condition A(−T₀)=0, representing an un-glycated state at the start of development in the bone marrow. This model, which accounts for the saturation of available glycation sites (the (1−A(τ)) term), is a more accurate representation than simpler linear or exponential models.

The parameters k_{g} and T₀ must be determined (Claims 4, 5).

The glycation constant k_{g} (Claim 4) may be determined from: (a) a pre-determined value from scientific literature; (b) an in vitro method, e.g., incubating RBCs of known initial HbA1c (A₁) in a solution of known glucose (G₁) for a time (T₁) to measure a final HbA1c (A₂) and calculating k_{g} (e.g., k_{g}≈(A₂−A₁)/(T₁*G₁*(1−(A₁+A₂)/2))); or (c) an in vivo method, e.g., administering tagged RBCs of known initial HbA1c (A₃) to a subject, retrieving them after a time (T₂) to measure the final HbA1c (A₄), and calculating k_{g} based on the subject's eAG (e.g., k_{g}≈(A₄−A₃)/(T₂*eAG*(1−(A₃+A₄)/2))).

The pre-circulation residence time T₀ (Claim 5) may be determined by a process comprising: (a) applying an identifiable stimulus to an RBC cohort in the bone marrow at a start time (T_start), such as (i) administering a traceable precursor (e.g., ⁵⁹Fe, ¹⁴C-glycine) or (ii) administering an erythropoietic stimulant (e.g., erythropoietin, EPO); (b) monitoring circulating peripheral blood to detect the first appearance of said RBC cohort at an end time (T_{end}); and (c) defining T₀ as the time difference T_{end}−Tₛₜₐᵣₜ.

### 3. METHOD 1: TRAJECTORY RECONSTRUCTION

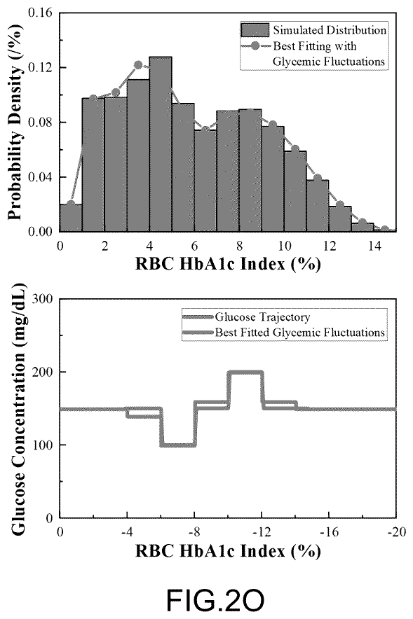

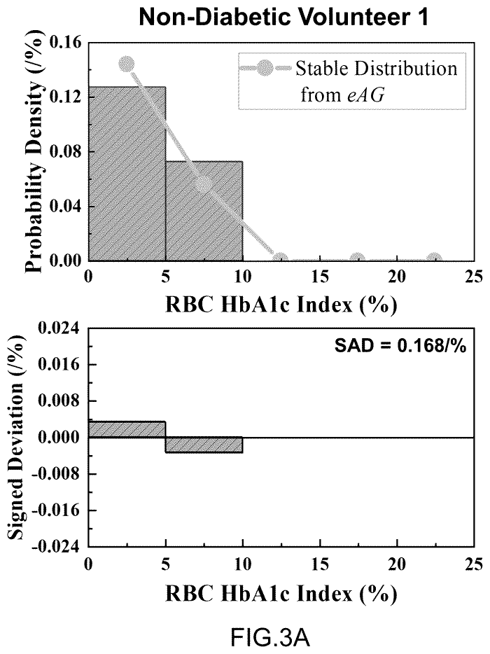

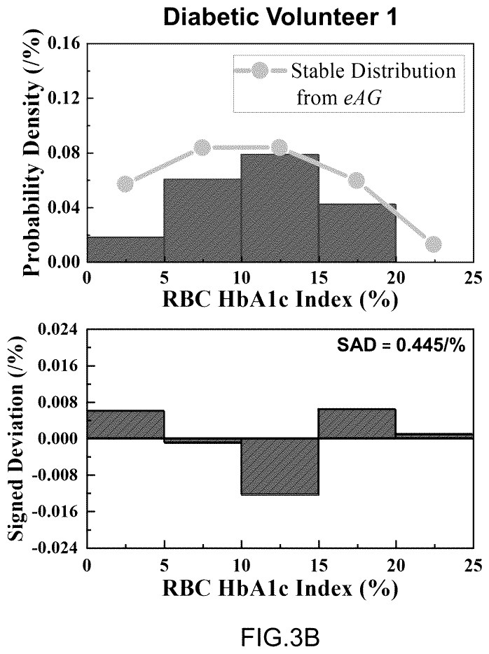

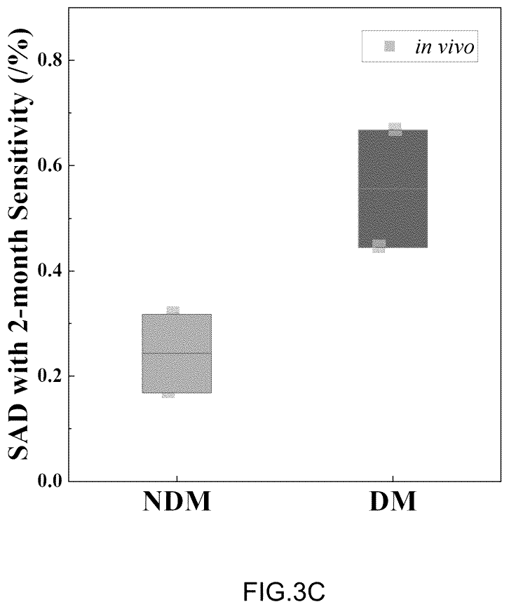

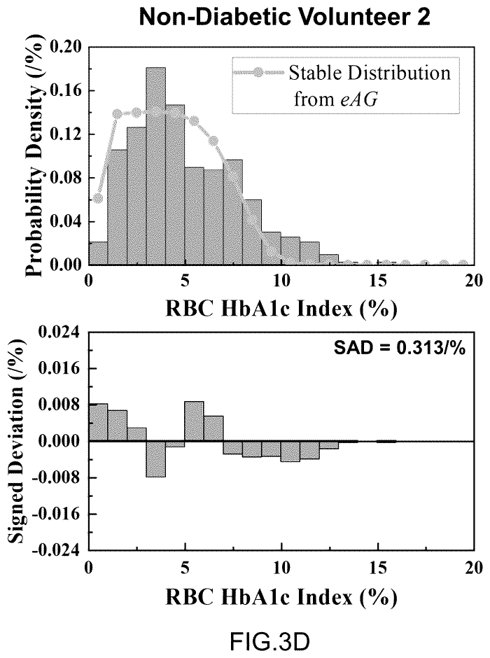

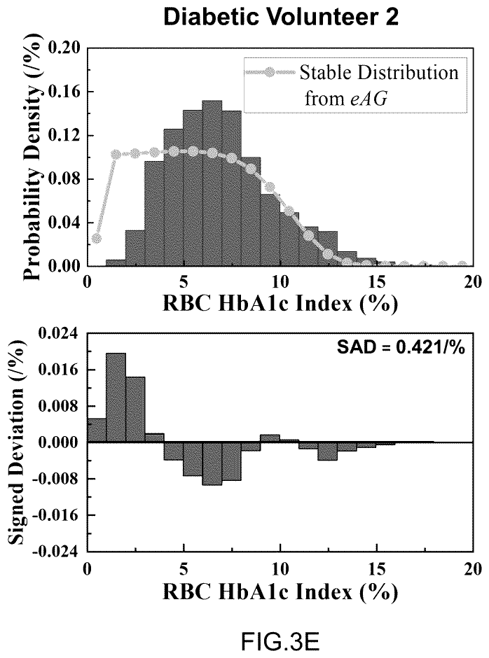

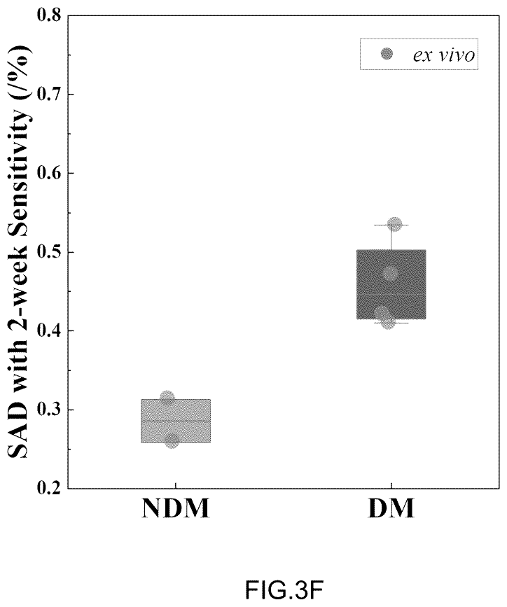

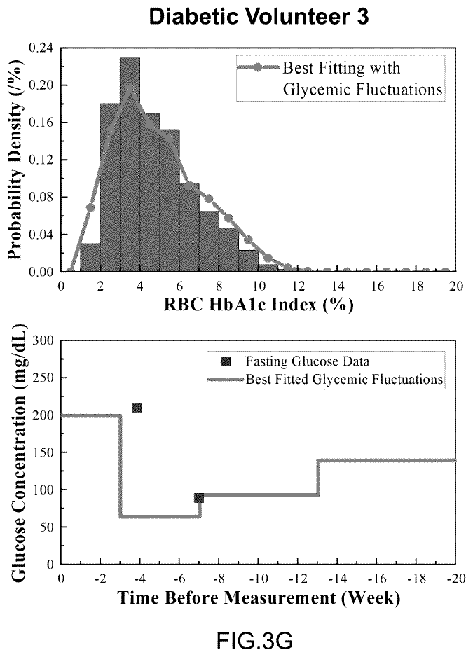

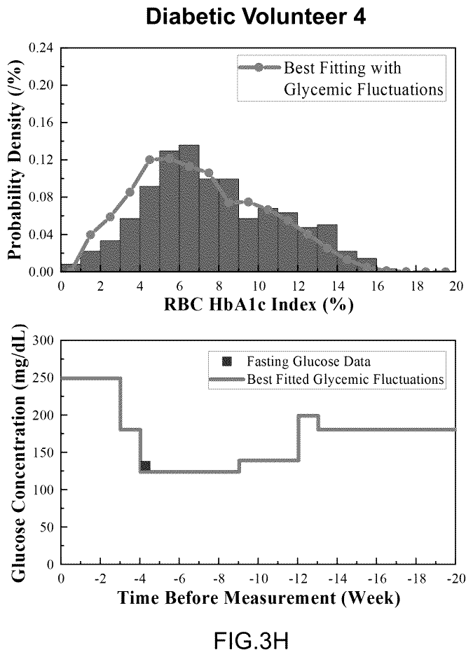

In the first analysis path, the system reconstructs the historical trajectory G(τ) (Claim 1). This is illustrated in FIG. 2O, FIG. 3G-3H.

Step (f): The system generates a reference RBC HbA1c distribution (FIG. 1, step 14, referred to as “third HbA1c distribution”). This begins by establishing an initial candidate glucose trajectory, G(τ). This initial candidate may be a simple, stable trajectory based on the subject's eAG.

The estimated average glucose (eAG) (Claim 7) can be determined from various sources, including, but not limited to: an average of the measured single-RBC HbA1c fractions {Aᵢ}, a conventional whole-blood HbA1c measurement, a data log from a continuous glucose monitoring (CGM) device, or an average of one or more fasting blood glucose measurements. In one non-limiting example, the eAG is calculated from the average whole-blood HbA1c fraction (F₀) using the empirical formula eAG=28.7*F₀(%)−46.7 (mg/dL).

With this candidate G(τ), the system solves the glycation model A(t) (from Claim 3) to get the mapping of age-to-HbA1c. It then computationally transforms the known PDF_{Age}(t) (from Claim 6) into the reference PDF_{HBA1c}(A) distribution.

Step (g): The system iteratively modifies the candidate G(τ). This is done by:
(i) Calculating a deviation metric between the first (measured) distribution and the reference (simulated) distribution. This deviation metric (Claim 10) can be any mathematical function quantifying dissimilarity, such as a Kolmogorov-Smirnov (KS) distance, an L1 norm (Sum of Absolute Deviations, as in FIG. 2N), an L2 norm (Root Mean Square Error), or an Earth-Mover Distance (EMD).
(ii) Adjusting the values of G(τ) within the discrete time segments (defined by ΔT) based on this deviation, in a direction that minimizes the metric. For example, if the measured distribution has more cells in a bin than the reference distribution (see FIG. 2N, bins 3-5), it implies the actual glucose was lower during that corresponding time period than in the candidate trajectory, so the G(τ) value for that period is adjusted downward.
(iii) To stabilize this reconstruction against noise, a mathematical regularization function (e.g., Tikhonov regularization or Total Variation regularization) may be applied to the candidate G(τ) during the modification step to enforce smoothness (Claim 11).
(iv) Repeating steps (f) and (g) until the deviation metric is below a predefined threshold (e.g., the maximum fitting error a is not reached).

Step (h): The final candidate G(τ) from the iterative process is output as the reconstructed historical glucose concentration trajectory. This is shown in FIG. 1 as “long-term blood glucose concentration trend”. This trajectory, G(τ), may be stored in a subject's electronic data record for clinical review or longitudinal follow-up (Claim 19). FIG. 2O, FIG. 3G, and FIG. 3H show examples of such outputted trajectories.

### 4. METHOD 2: GLYCEMIC VARIABILITY (GV) ASSESSMENT

In the second analysis path, the system assesses the subject's GV (Claim 2). This is illustrated in FIG. 2M-2N and FIG. 3A-3F.

Step (e): The system determines an estimated average glucose (eAG) level for the subject, using one of the methods described in Claim 7 (e.g., from a whole-blood HbA1c measurement).

Step (f): The system generates a second RBC HbA1c distribution (FIG. 1, step 13). This is done by: (i) establishing a stable glucose trajectory, where G(τ) is held constant at the eAG value (see FIG. 2M, gray line); (ii) solving the glycation model A(t) (from Claim 3) based on this stable trajectory; and (iii) computationally transforming the PDF_{Age}(t) (from Claim 6) into this second, stable distribution (see FIG. 2N, gray line).

Step (g): The system calculates a deviation metric (Claim 10) representing the absolute deviation between the first (measured) distribution (FIG. 2N, purple bars) and the second (stable) distribution (FIG. 2N, gray line). An example of this deviation is shown in FIG. 2N and FIG. 3A, 3B, 3D, 3E. The metric may be, for example, the Sum of Absolute Deviations (SAD).

Step (h): This deviation metric is output as a quantitative indicator of the subject's glycemic variability (output of step 13 in FIG. 1, “glucose variability”). A low deviation (as in FIG. 3B, 3G) indicates a stable glycemic history, while a high deviation (as in FIG. 3A, 3D) indicates significant glycemic variability. As shown in FIG. 3C and FIG. 3F, this GV metric can effectively distinguish between diabetic and non-diabetic cohorts. This GV score may be output to a display or an electronic data record (Claim 20).

### 5. SYSTEM EMBODIMENTS

The invention is also embodied as a system (Claim 14) comprising a data processing terminal with processors and a non-transitory machine-readable medium (e.g., software) storing instructions. When executed, these instructions cause the system to perform the core steps of (i) receiving the first distribution, (ii) defining the age distribution, (iii) defining the glycation model, and (iv) generating a subsequent distribution.

This system can be configured to perform the full method of trajectory reconstruction (Claim 15, implementing method of Claim 1) or the full method of GV assessment (Claim 16, implementing method of Claim 2).

The preferred embodiment(s) herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method for reconstructing a subject's historical glucose concentration trajectory, G(τ), from single-cell glycated hemoglobin (HbA1c) measurements, the method comprising:
(a) obtaining a plurality of single-red-blood-cell (RBC) HbA1c fractions, {Aᵢ}, from a total of N RBCs sampled from a subject, wherein each said HbA1c fraction (A) represents a ratio of a glycated hemoglobin amount to a total hemoglobin amount within a single RBC;
(b) generating a first RBC HbA1c distribution, PDF_{HbA1c}(A), from said plurality of {Aᵢ}, said first distribution being represented as a histogram with a selected bin width ΔA, wherein said ΔA is dependent on said N;
(c) defining a whole-blood RBC age probability density function, PDF_{Age}(t), representing a statistical distribution of RBCs having a circulation age t, wherein t is the time since release from bone marrow;
(d) defining an RBC glycation model, A(t), that provides a deterministic mapping between said historical glucose concentration trajectory, G(τ), and a corresponding HbA1c fraction for an RBC of age t;
(e) determining a time resolution ΔT corresponding to said bin width ΔA, wherein said ΔT defines discrete segments for said G(τ);
(f) generating a reference RBC HbA1c distribution by: (i) establishing an initial candidate glucose trajectory, G(τ); (ii) solving said glycation model A(t) based on said candidate G(τ); and (iii) computationally transforming said PDF_{Age}(t) into said reference RBC HbA1c distribution using the mapping provided by A(t);
(g) iteratively modifying said candidate G(τ) by: (i) calculating a deviation metric between said first RBC HbA1c distribution and said reference RBC HbA1c distribution; (ii) adjusting values of G(τ) within said discrete segments based on said deviation metric to create a new candidate G(τ); and (iii) repeating steps (f) and (g) until said deviation metric is below a predefined threshold; and
(h) outputting the final candidate G(τ) from step (g) as the reconstructed historical glucose concentration trajectory for the subject.

2. The method of claim 1, wherein said RBC glycation model A(t) is defined by the integral equation:
$$A ( t ) = {\int }_{- {T}_{0}}^{t} {k}_{g} * G ( τ ) * ( 1 - A ( τ ) ) d τ .$$
wherein k_{g} is a hemoglobin glycation reaction constant, T₀ is a pre-circulation residence time in bone marrow, G(τ) is the glucose concentration at time τ, and an initial condition A(−T₀) is zero.

3. The method of claim 2, wherein said k_{g} is a constant determined from a source selected from the group consisting of:
(a) a pre-determined value obtained from a scientific literature reference;
(b) an in vitro method comprising incubating RBCs of a known initial HbA1c fraction (A₁) in a solution of known glucose concentration (G₁) for a time (T₁) to measure a final HbA1c fraction (A₂) and calculating said k_{g}; and
(c) an in vivo method comprising administering tagged RBCs of a known initial HbA1c fraction (A₃) to said subject, retrieving said tagged RBCs after a time (T₂) to measure a final HbA1c fraction (A₄), and calculating said k_{g} based on an estimated average glucose (eAG) of said subject.

4. The method of claim 2, wherein said T₀ is determined by a process comprising:
(a) applying an identifiable stimulus to an RBC cohort in bone marrow at a start time (Tₛₜₐᵣₜ), wherein said stimulus is selected from the group consisting of: (i) administering a traceable precursor, said precursor being ⁵⁹Fe or ¹⁴C-glycine, and (ii) administering an erythropoietic stimulant, said stimulant being erythropoietin (EPO);
(b) monitoring circulating peripheral blood to detect a first appearance of said RBC cohort at an end time (T_{end}); and
(c) defining T₀ as the time difference between T_{end} and Tₛₜₐᵣₜ.

5. The method of claim 1, wherein said whole-blood RBC age probability density function, PDF_{Age}(t), is defined by a method selected from the group consisting of: (a) direct measurement of RBC ages from said subject; and (b) reconstructing said PDF_{Age}(t) by applying a mathematical survival function model to a measured survival rate, wherein said survival rate is obtained by (i) administering a tagged cohort of RBCs, said tagged cohort being tagged by biotin or a radioactive element, to said subject, (ii) sampling blood at a plurality of different time points, and (iii) measuring a change in the tagged RBC population over time.

6. The method of claim 3, wherein said estimated average glucose (eAG) is determined from at least one data source selected from the group consisting of: an average of said {Aᵢ}, a whole-blood HbA1c measurement, a continuous glucose monitoring data log, and an average of one or more fasting blood glucose measurements.

7. The method of claim 1, wherein said selected bin width ΔA is determined as a function of said N by applying a statistical histogram binning rule to said first RBC HbA1c distribution, PDF_{HBA1c}(A).

8. The method of claim 1, wherein said time resolution ΔT is determined as a function of said selected bin width ΔA and an estimated glycation rate, said glycation rate being derived from said RBC glycation model A(t) and said historical glucose concentration trajectory, G(τ).

9. The method of claim 1, wherein said deviation metric comprises any mathematical function that quantifies a dissimilarity between said first RBC HbA1c distribution and said subsequent (reference or second) RBC HbA1c distribution.

10. The method of claim 1, wherein said step (g) of iteratively modifying said candidate G(τ) further comprises applying a mathematical regularization function to said G(τ) to enforce smoothness and stabilize the reconstruction against noise.

11. The method of claim 1, wherein said step (a) of obtaining said plurality of {Aᵢ} is performed by an assay or technique capable of determining a glycated hemoglobin (HbA1c) fraction within a single red blood cell, said assay or technique including, but not limited to, High-Performance Liquid Chromatography (HPLC), capillary electrophoresis, immunoassay, mass spectrometry, Raman-based methods, transient absorption microscopy, absorption spectroscopy, or optical microscopy.

12. The method of claim 1, wherein said step (a) of obtaining said plurality of {Aᵢ} is performed by Resonant-Enhanced Color-Resolved Third-Harmonic-Generation (RE-cTHGM) microscopy, said microscopy method comprising:
(a) exciting a single red blood cell with single broadband laser beam or wavelength-tunable laser beam;
(b) generating a color-resolved third-harmonic-generation (THG) signal from said red blood cell, wherein said THG signal is resonantly enhanced based on the distinct absorption spectra of hemoglobin (Hb) and glycated hemoglobin (HbA1c);
(c) partitioning said resonantly enhanced THG signal into at least two distinct spectral channels; and
(d) computing said HbA1c fraction {Aᵢ} for said single red blood cell from a relative signal intensity between said at least two spectral channels, wherein a first spectral channel is selected to be more sensitive to non-glycated hemoglobin (Hb) and a second spectral channel is selected to be more sensitive to glycated hemoglobin (HbA1c).

13. A system for analyzing a subject's historical glycemic status, the system comprising: (a) a data processing terminal comprising one or more processors; and (b) a non-transitory machine-readable medium operatively coupled to said one or more processors, said medium storing instructions that, when executed, cause said data processing terminal to perform at least the following steps: (i) receiving a first RBC HbA1c distribution, PDF_{HBA1c}(A), generated from a plurality of HbA1c fractions, {Aᵢ}, obtained from N RBCs; (ii) defining a whole-blood RBC age probability density function, PDF_{Age}(t); (iii) defining an RBC glycation model, A(t), that provides a deterministic mapping between a historical glucose concentration trajectory, G(τ), and a corresponding HbA1c fraction; and (iv) generating a subsequent RBC HbA1c distribution based on said PDF_{Age}(t) and said A(t).

14. The system of claim 13, wherein said instructions, when executed, further cause said data processing terminal to perform the method of claim 1.

15. The system of claim 13, wherein said instructions, when executed, further cause said data processing terminal to perform the method of claim 2.

16. The system of claim 13, further comprising a data acquisition interface configured to receive said plurality of {Aᵢ} from an external assay.

17. The system of claim 16, wherein said external assay comprises a Resonant-Enhanced Color-Resolved Third-Harmonic-Generation (RE-cTHGM) instrument operatively coupled to said data acquisition interface.

18. The method of claim 1, further comprising storing said reconstructed historical glucose concentration trajectory, G(τ), in a subject's electronic data record for clinical review or longitudinal follow-up.
